(19) United States
(12) Patent Application Publication
(10) Pub. No.: US 2026/0165715 A1
(43) Pub. Date: Jun. 18, 2026

(54) DRILL GUIDE AND ARRAY CLAMP

(71) Applicant: DEPUY IRELAND UNLIMITED COMPANY, Ringaskiddy (IE)

(72) Inventors: Adam Ryals, Ringaskiddy (IE); Travis Crowe, Ringaskiddy (IE); Bryan Rose, Ringaskiddy (IE)

(73) Assignee: DEPUY IRELAND UNLIMITED COMPANY, Ringaskiddy (IE)

(21) Appl. No.: 18/985,613

(22) Filed: Dec. 18, 2024

(51) Int. Cl. A61B 17/17

(52) U.S. Cl. CPC A61B 17/1725

(57) ABSTRACT

A drill guide clamp for attachment to a bone may include a worm screw, a sliding rack moveably connected to the worm screw, and at least two circular integrated drill tubes that extend through the drill guide clamp. Each drill tube may be sized to allow at least one screw or pin to fix the drill guide clamp to the bone. The drill tubes may be positioned to ensure that the pins or screws that fix the drill guide clamp to the bone are parallel. The sliding rack may include engagement interfaces that are moveable between an engagement and non-engagement position with the pins or screws via movement of the sliding rack.

### BACKGROUND

This disclosure relates to orthopedic fixation systems for surgery and more particularly to a drill guide and plate attachment mechanism for facilitating orthopedic surgical plating procedures.

Surgical plates in various forms have been used by orthopedic surgeons to fixate bones in a specifically desirable fashion or position such that bone knitting or healing occurs between the juxtaposed bony elements. In this regard, plating is employed across bony fracture sites or across surgical fusion sites to fixate and hold the bone components in a preferred configuration until solid bony union occurs. Because independent movement of the bony elements relative to each other retards or prevents bony union from occurring, bone fixation is frequently required, much like an external cast is used to inhibit motion sufficient to allow healing of a broken arm or leg bone.

The plate allowing fixation of the bony components is generally secured to the bone itself by the use of specifically designed bone screws. Drilling and tapping of bone is frequently required to allow the screws or pins to be appropriately placed.

The ridged attachment of markers to the bony anatomy consists of multiple steps and is typically comprised of a construct that includes Schanz pins drilled into the bone and an external fixation device connecting the pins to the tracker. Maintaining stable fixation of the array construct through the case can be challenging. Several failure mechanisms have been identified in lab and operating room settings. These failure mechanisms include: 1) pins that are not parallel can result in a toggling between two different stable positions, 2) inadequate clamping force leading to movement of the fixation base relative to the pins, 3) Hirth coupling failure due to tapered teeth being compressed on top of one another and later meshing if a small force is applied resulting in loose or unstable fixation, 4) array clamp hinge impingement with fixation base prevents tightening of Hirth coupling, 5) Excessively long moment arm between the locking construct and the pin/bone interface and/or 6) pin bone interface failure due to continuous external pressure.

There is a need for improved systems and devices that create a robust, ridged and locked construct that is easily inserted and removed for temporary bony fixation and support.

### SUMMARY

The present application relates to drill guide clamps for attachment to a bone. The drill guide clamp may include a worm screw, a sliding rack moveably connected to the worm screw, and a plurality of circular openings that extend through the drill guide clamp. Each circular opening of the plurality of circular openings may be sized to allow a respective screw or a pin to fix the drill guide clamp to the bone. The plurality of circular openings may be positioned to ensure that the respective screws or the pins that fix the drill guide clamp to the bone are parallel. The design configuration can be adjusted based on the diameter of the intended pins or screws.

The sliding rack may include engagement interfaces that are moveable between an engagement and non-engagement position with the pins or screws via movement of the sliding rack.

The worm screw may include a knob configured to move the sliding rack via movement of the worm screw. The knob may comprise a shaft that contacts the worm screw. The worm screw may mesh with groves of the sliding rack. Movement of the knob may rotate the worm screw causing movement of the sliding rack.

Cleaning of the drill guide clamp allows it to have further use. To accommodate cleaning, the knob may be separable from the worm screw to permit disassembly.

The drill guide clamps may include a spring mechanism configured to apply a constant pressure to the screws or the pins once the screws for easy insertion and minimal advancement of the screw and rack construct for rapid fixation. In some embodiments, the spring mechanism is located between the knob and the worm screw.

The plurality circular opening and sliding rack may be configured to allow insertion of the screws or the pins into the bone to secure the drill guide clamp to the bone prior to surgery.

The drill guide clamp may include an attachment feature to attach a further element, such as the navigational tracker, to the drill clamp guide. The navigational tracker may indicate the position of the drill guide clamp to a computer during computer assisted surgery. For example, the attachment feature may include a locking ball and socket.

Some or all of the circular openings may comprise a drill tube. In some embodiments, each circular opening of the plurality circular openings may be threaded to allow for the drill tube to be replaced with a drill tube of a different size, shape, or thread type. The threaded guides allow for the attachment of multiple length soft-tissue guide tubes to both protect the soft-tissue and define a fixed yet adjustable distance from the bone surface.

The sliding rack may be configured to move between the engagement and non-engagement position in response to rotations of the worm screw.

A system may include a drill guide clamp. For example, the system may include a worm screw, a sliding rack moveably connected to the worm screw, and a plurality of circular openings that extend through the drill guide clamp. Each circular opening of the plurality of circular openings may be sized to allow a respective screw or a pin to fix the drill guide clamp to the bone. The plurality of circular openings may be positioned to ensure that the respective screws or pins that fix the drill guide clamp to the bone are parallel and optimally positioned for simultaneous engagement by a single locking element. In some examples, the system may also include a navigational tracker that includes a navigational array.

The system may include a surgical assistance system that includes a processor that is configured to track the position of the navigation array to determine a position of the system during a surgical procedure. The surgical assistance system may comprise any combination of a camera, a robotic system, a virtual reality system, and/or an augmented reality system. Other features of the drill guide clamps and systems may be included, for example, as described herein.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings.

FIG. 1 illustrates a cross-section drawing of an example drill guide assembly according to some clamps of the art.

FIG. 2 illustrates an example drill guide that includes a worm screw, integrated drill tubes for bone fixation, and a tracker device.

FIG. 3 illustrates an example drill guide clamp showing details of the worm screw, sliding rack and spring mechanism.

FIG. 4 illustrates a cross-sectional schematic of an example drill guide assembly.

FIG. 5 illustrates a closeup of a portion of the drill guide clamp that can move between an open and closed position.

FIG. 6 illustrates a view of a drill guide clamp where Schanz pins are inserted into the circular openings.

FIG. 7 illustrates additional detail of various components of one example of a drill guide clamp.

### DETAILED DESCRIPTION

The present disclosure, in some embodiments, thereof, relates to drill guide assemblies that may be used in, but not exclusively, in bone fixation.

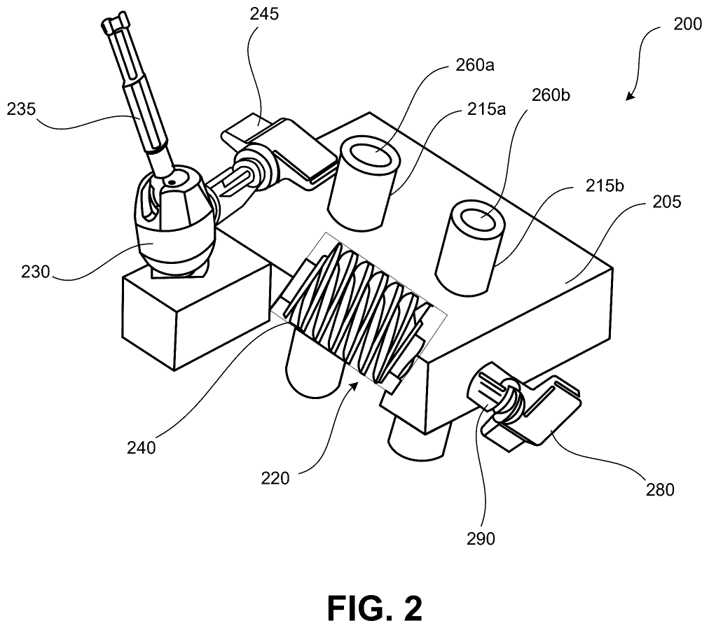

In some aspects, the disclosure concerns drill guide clamps that can be used for attachment to a bone. In certain embodiments, the drill guide clamps include a worm screw; a sliding track moveably connected to the worm screw; integrated drill tubes that extend through the drill guide clamp designed to allow a plurality of screws or pins to fix the drill guide clamp to the bone, the drill tubes ensure that the pins or screws that fix the drill guide clamp to the bone are parallel; and the sliding track having engagement interfaces that are moveable between an engagement and non-engagement position with the pins or screws via the sliding track. In some embodiments, the worm screw comprises square threads. These features are illustrated in FIGS. 2-4. Some components of the fixation system may be made of metal, such as steel, titanium, or the like to reduce wear and allow reuse of the drill guide clamp.

The drill guide clamps (also referred to drill guide and array clamps) can provide a compact design that improves process efficiency and consistency while reducing the potential of instrument damage.

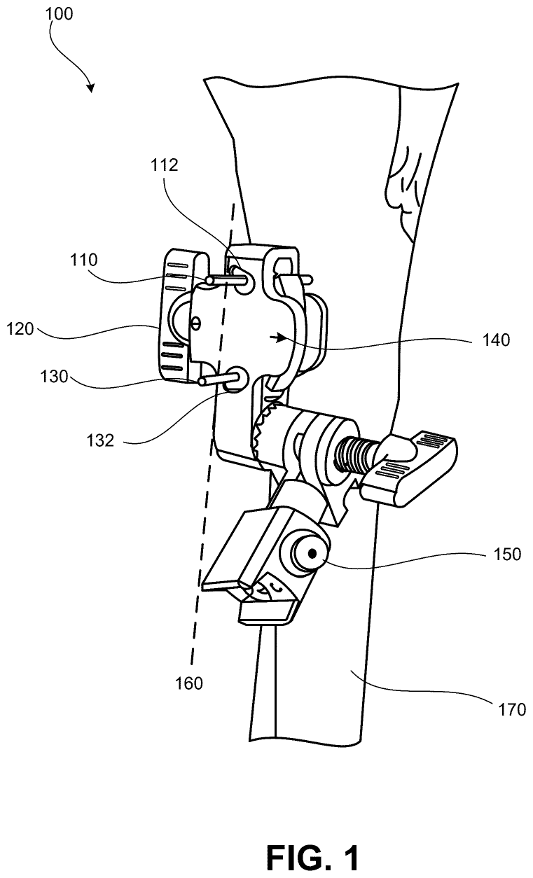

As background, FIG. 1 shows an example of a prior art drill guide clamp 100 attached to a tibial bone 170. A first array drill pin 110 and a second array drill pin 130 are shown attaching the drill guide clamp 100 to the tibial bone 170. The first and second array drill pins 110, 130 may pass through first and second openings 112, 132 of the drill guide clamp 100, respectively. The first and second array drill pins 110, 130 are aligned with the tibial long axis 160. The first and second openings 112, 132 are oval in shape. The drill guide clamp 100 may include a pin wingnut 120 that may be used to lock or unlock the position of a navigation tracker (not shown) that may be utilized in computer assisted surgery. FIG. 1 shows an arrow 140 pointing towards a camera (not shown) that may monitor the location of the navigation tracker during the surgery. The drill guide clamp 100 may include a button 150 that, when compressed, allows for attachment/detachment of the navigation tracker. For operability purposes, the button 150 should be positioned facing away from the bone.

As way of further background, a variety of surgical procedures utilize surgical navigation or tracking to assist in positioning surgical instruments relative to portions of the anatomy of a patient during a procedure. One such type of procedure is a robotic or robot-assisted surgical procedure, where surgical navigation can be important to correctly position a robotically controlled or assisted surgical instrument relative to a patient.

There are several known surgical navigation or tracking technologies, including optical navigation or tracking systems that utilize, e.g., stereoscopic sensors to detect infra-red (IR) or other light reflected or emitted from one or more optical markers affixed to surgical instruments and/or portions of a patient's anatomy. By way of further example, a tracker having a unique constellation or geometric arrangement of reflective elements can be coupled to a surgical instrument and, once detected by stereoscopic sensors, the relative arrangement of the elements in the sensors'field of view, in combination with the known geometric arrangement of the elements, can allow the system to determine a three-dimensional position and orientation of the tracker and, as a result, the instrument or anatomy to which the tracker is coupled.

In known surgical navigation technologies, a navigation array or tracker can be mounted on an instrument that is received and/or controlled by a robotic arm to identify the position of the instrument. In some instances, a navigation array or tracker can be formed integrally with the instrument itself. In other instances, a navigation array can be removably attached to an instrument and can be used to track the position of multiple instruments over the course of a surgical procedure. Examples of computer assisted surgical navigation techniques and navigational trackers can be found, for example, in U.S. Pat. Nos. 11,944,391 and 12,016,641, which are incorporated herein by reference.

In some cases, the technology may comprise computer aided surgery (CAS) comprising an augmented reality (AR) system configured to display augmented reality information, a position tracking system configured to track positions of objects, an instrument coupled to a navigational tracker detectable by the position tracking system, and a controller configured to determine a position of the instrument, based on the determined position, display augmented reality information using the AR system, the augmented reality information comprising a representation of a relationship between at least a distal end of the instrument and tissue of the patient, and if the instrument moves to a second position, updating the representation. See, for example, published U.S. Patent Application No. 2023/293,259, which is incorporated herein by reference.

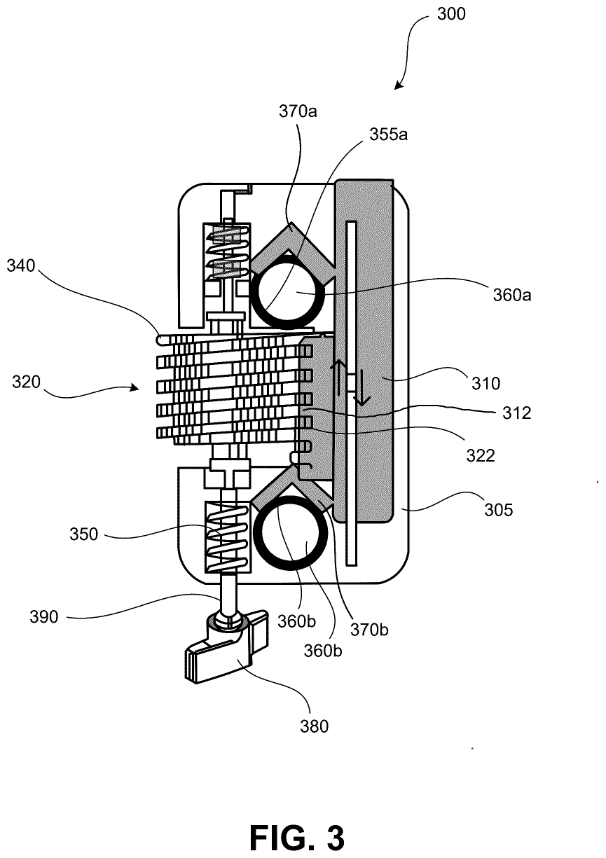

FIG. 2 shows an example of a drill guide clamp 200 that, for example, may provide improved functionality as compared to the drill guide clamp 100. The drill guide clamp 200 may be configured to be attached to a bone. The drill guide clamp 200 may include a body 205, at least two drill tubes 215a, 215b that include at least two circular openings 260a, 260b, respectively, a worm screw 220 having threads 240, a knob 280,, a shaft 290, and a sliding rack (not shown). The worm screw 220 is a type of gear arrangement that interacts with the teeth of a gear (such as the sliding rack depicted in FIG. 3 as 310). In the instant disclosure, a sliding rack 310 is moveably connected to the worm screw by threads with rack teeth (depicted in FIG. 3 as 312) of the sliding rack.

The least two circular openings 260a, 260b may extend through the body 205 of the drill guide clamp 200. The least two circular openings 260a, 260b may be formed through at least two drill tubes 215a, 215b that extend through the body 205 the drill guide clamp 200. Each circular opening 260a, 260b may be sized to allow at least one screw or pin (e.g., the first array drill pin 110 and the second array drill pin 130 of FIG. 1) to extend through the body 205 of the drill guide clamp 200 to fix the drill guide clamp 200 to the bone.

The circular opening 260a, 260b of the drill tubes 215a, 215b may be positioned to ensure that the pins or screws that fix the drill guide clamp 200 to the bone are parallel to one another when the screws or pins couple the drill guide clamp 200 to the bone. By ensuring that the screws or pins are parallelly spaced from one another, the drill guide clamp 200 that has the circular opening 260a, 260b may improve stability of the drill guide clamp 200 (e.g., and any navigation trackers attached thereto) relative to the bone during surgery.

In some examples, the circular openings 260a, 260b of the drill tubes 215a, 215b are threaded to allow for a screw to pass through the circular openings 260a, 260b. Any suitable type of pin or screw may be utilized. In one embodiment, the screw is a Schanz screw. Further, in some examples, the drill guide clamp 200 may include circular openings 260a, 260b through the body 205 of the drill guide clamp 200, and drill tubes 215a, 215b of different shapes or sizes may be attached to the circular openings 260a, 260b depending on the size and/or thread type needed for the use. The variable size and/or thread type may allow the surgeon to account for variability in patient and bone size while keeping the overall construct as close to the bone surface as possible. In such examples, the circular openings 260a, 260b may include threads on the outside of the circular openings 260a, 260b, and the interchangeable drill tubes 215a, 215b may include threads on the inside of the drill tubes 215a, 215b so the drill tubes 215a, 215b can be screwed onto the circular openings 260a, 260b to secure the drill tubes 215a, 215b to the drill guide clamp 200.

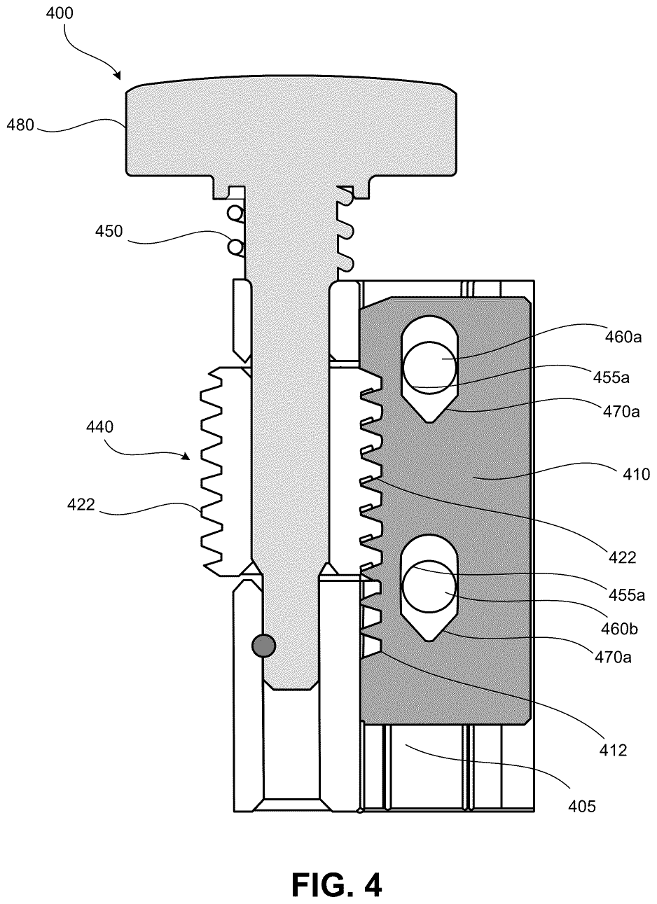

The sliding rack (not shown) may include an engagement interface that is configured to engage the pins or screws to an interior sidewall of the circular openings 260a, 260b (e.g., as shown in FIGS. 3 and 4). For example, the sliding rack may be configured to be moved between a non-engagement position where the engagement interface of the sliding rack does not secure the pins or screws to the drill guide clamp 200, and an engagement position where the engagement interface of the sliding rack does secure the pins or screws to the drill guide clamp 200. For example, when the drill guide clamp 200 is in the proper position for surgery, the pins or screws could be placed through the circular openings 260a, 260b when the sliding rack is in the non-engagement position. Then, once in place, the sliding rack can be moved into the engagement position so that the engagement interface of the sliding rack secures the drill guide clamp 200 to the pins or screws.

The sliding rack may be moveably connected to the worm screw 220 which is configured to move the sliding rack between the engagement position and the non-engagement position. For example, the knob 280 may be configured to be rotated clockwise to move the screw 220 to cause the engagement interface of the sliding rack to move into the engagement position, and the knob 280 may be configured to be rotated counter-clockwise to move the worm screw 220 to cause the engagement interface of the sliding rack to move into the non-engagement position. The knob 280 may be attached to the worm screw 220 by the shaft 290. In some embodiments, the worm screw 220 and the shaft 290 may be consolidated into a single construct. The circular opening 260a, 260b may be positioned to ensure that the sliding rack can be moved between an engagement and non-engagement position.

In some examples, the worm screw 220 may include square threads. The square threads of the worm screw 220 may increase the clamping force providing by the engagement interface of the sliding rack on the pines or screw. The square threads may be robust to damage. In addition, the worm screw 220 may be strong and/or may not allow pressure to reduce over time. Moreover, the worm screw 220 may provide increased force feedback relative to prior art drill guide clamps, which ensures the user applies adequate turning force to achieve stable fixation by hand and/or can reduce the likelihood that users will damage the drill guide clamp by over torquing the drill guide clamp.

The benefits of square threads, however, can be offset by the number of turns needed to advance the clamping construct, which reduces the procedure's efficiency. To overcome this issue, the drill guide clamp 200 may minimize the distance that the sliding rack must travel to achieve full compression. For instance, the drill guide clamp 200 may include a spring mechanism (e.g., a spring mechanism 350, as illustrated in FIG. 3) that can allow the rack to apply a continuous force to the rack allowing it to slide into place and can minimize the number of turns needed. The spring mechanism can continuously apply slight pressure to the pins once fully inserted into the guide clamp. This ensures the minimum number of turns required to applying major compressive locking force to engage a sliding rack against the pins fully.

In robotic/computer-assisted surgery, there is a need to determine the real-time positions of objects through ridged fixation of markers used to track instruments and patient anatomy. In certain embodiments, the drill guide clamp 200 may include a tracker device 235 (e.g., a navigation tracker). The tracker device 235 can be attached to a mounting mechanism such as a locking ball and socket mount 230. A locking ball and socket mount 230 can enhance the flexibility of motion between the connection of the tracker device 235 and the drill guide clamp 200. The drill guide clamp 200 may include a locking knob or handle 245 that is used to fix the tracker device 235 to the ball and socket mount 230. While the system is illustrated by the locking ball and socket mount 230, other appropriate attachment devices may be utilized to fix the tracker device 235 to the drill guide clamp 200. For example, the tracker 235 may be an integral part of the drill guide clamp 200 or attached by a clamp.

Multiple computer-assisted surgery methods exist. In some embodiments, a tracker device is used to register the target plane with a coordinate system attached to the anatomical structure to be cut or manipulated, using images or geometric patient data collected during surgery.

The tracker device 235 may be a passive tracker which allows identification of the location of the drill guide clamp via scanning of the area comprising the tracker. This form of tracker device may utilize optical, electromagnetic, or other appropriate technology. For example, the passive marker, such as a reflective marker, that can be detected by at least one sensor or camera of the computer-assisted surgery system without actively communicating with the computer of the computer-assisted surgery system. Other trackers may be infrared light emitting diodes (LEDs).

In some examples, intra-operative images or data are used to register pre-operative images in a unique coordinate system attached to the anatomical structure, and usually represented by a tracker that can use computer assisted surgery technologies. In such examples, a tracker device 235 with electronic transmitters can be utilized. In certain embodiments, the transmitters may incorporate both receivers and transmitters so that the markers can receive and transmit a signal. The transmitters may be capable of receiving and sending signals with an external device, such as, for example, a detection device, which will be described in greater detail below. Alternatively, and/or in addition, the tracker device 235 may be capable of receiving and sending signals with respect to one another in order to determine their relative orientation. The signal preferably contains information as to the position and/or orientation (collectively referred to herein as orientation) of the tracker device 235 and hence the attached bone.

FIG. 3 presents an example of a drill guide clamp 300. In some embodiments, the drill guide clamp 300 may be an example of the drill guide clamp 200. The drill guide clamp 300 may include a worm screw 320, a sliding rack 310 moveably connected to the worm screw 320, and at least two circular openings 360a, 360b that extend through a body 305 of the drill guide clamp 300. The worm screw 320 comprises a plurality of threads 322. The sliding rack 310 comprises a plurality of rack teeth 312 that are sized to receive the threads 340 of the worm screw 320.

The least two circular openings 360a, 360b may extend through the body 305 of the drill guide clamp 300. The openings 360a, 360b may define respective inner surfaces 355a, 355b. The least two circular openings 360a, 360b may be formed through at least two drill tubes that extend through the body 305 the drill guide clamp 300. Each circular opening 360a, 360b may be sized to allow at least one screw or pin (e.g., the first array drill pin 110 and the second array drill pin 130 of FIG. 1) to extend through the body 305 of the drill guide clamp 300 to fix the drill guide clamp 300 to the bone.

The circular opening 360a, 360b may be positioned to ensure that the pins or screws that fix the drill guide clamp 300 to the bone are parallel to one another when the screws or pins couple the drill guide clamp 300 to the bone. By ensuring that the screws or pins are parallelly spaced from one another, the drill guide clamp 300 that has the circular opening 360a, 360b may improve stability of the drill guide clamp 300 (e.g., and any navigation trackers attached thereto) relative to the bone during surgery.

In some examples, the circular openings 360a, 360b are threaded to allow for a screw to pass through the circular openings 360a, 360b. Any suitable type of pin or screw may be utilized. In one embodiment, the screw is a Schanz screw. Further, in some examples, the drill guide clamp 300 may include circular openings 360a, 360b through the body 305 of the drill guide clamp 300, and drill tubes of different shapes or sizes may be attached to the circular openings 360a, 360b depending on the size and/or thread type needed for the use. The variable size and/or thread type may allow the surgeon to account for variability in patient and bone size while keeping the overall construct as close to the bone surface as possible. In such examples, the circular openings 360a, 360b may include threads on the outside of the circular openings 360a, 360b, and the interchangeable drill tubes may include threads on the inside of the drill tubes so the drill tubes can be screwed onto the circular openings 360a, 360b to secure the drill tubes to the drill guide clamp 300.

The sliding rack 310 may include engagement interfaces 370a, 370b that are configured to engage the pins or screws to the respective inner surfaces 355a, 355b of the circular openings 360a, 360b, respectively. The engagement interfaces 370a, 370b are depicted as “L” shaped. As described in more detail herein, tightening the worm screw 320 may move the engagement interfaces 370a, 370b across the respective circular openings 360a, 360b to secure the drill guide clamp 300 to the pins or screws. Although illustrated as an “L” shape, other shapes that allow for the engagement and non-engagement of the sliding rack 310 with the pins or screws can be utilized. The engagement interfaces 370a, 370b may be part of the sliding rack 310 either in a one piece construction or via an attachment mechanism.

As described herein, the sliding rack 310 may be configured to be moved between a non-engagement position where the engagement interfaces 370a, 370b do not secure the pins or screws to the drill guide clamp 300, and an engagement position where the engagement interfaces 370a, 370b of the sliding rack 310 secure the pins or screws to the drill guide clamp 300. For example, when the drill guide clamp 300 is in the proper position for surgery, the pins or screws could be placed through the circular openings 360a, 360b when the sliding rack 310 is in the non-engagement position. Then, once in place, the sliding rack 310 can be moved into the engagement position so that the engagement interfaces 370a, 370b secure the drill guide clamp 300 to the pins or screws.

The engagement interfaces 370a, 370b may be configured to pressure and secure the screw or pin to allow for multiple points of contact with the screw or pin when the screw or pin is located in the respective circular openings 360a, 360b. In some embodiments, the engagement interfaces 370a, 370b allow for three points of pressure on the screws or pins. For example, when the sliding rack 310 in is the engaged position, the screw or pin may have at least one point of contact with inner surface 355a, 355b of the respective circular openings 360a, 360b, and may have at least two points of contact with the respective engagement interfaces 370a, 370b (e.g., one on either portion or leg of the “L” shape).

In some examples, the sliding rack 310 may slide within a track in the drill guide clamp 300 that, for example, may allow for a compact design that insures secure contact of the engagement interfaces 370a, 370b with the pins or screws.

The drill guide clamp 300 may include a worm screw 320. The drill guide clamp 300 may include a worm wheel or other moveable elements, such as the sliding rack 310. The worm screw 320 may have the appearance of a typical screw and may be configured to rotate according to its input (e.g., like movement of the knob 380 and shaft 390). The sliding rack 310 may be moved according to the rotation of the worm screw 320. This arrangement can transmit a high among of torque and engage the pins or screws used to affix the drill guide clamp 300 to a bone.

The sliding rack 310 may be moveably connected to the worm screw 320, and the worm screw 320 may be configured to move the sliding rack 310 between the engagement position and the non-engagement position. For example, the knob 380 may be configured to be rotated clockwise to move the worm screw 320 to cause the engagement interface of the sliding rack to move into the engagement position, and the knob 380 may be configured to be rotated counter-clockwise to move the worm screw 320 to cause the engagement interface of the sliding rack to move into the non-engagement position. The knob 380 may be attached to the worm screw 320 by the shaft 390. The circular opening 360a, 360b may be positioned to ensure that the sliding rack can be moved between an engagement and non-engagement position.

In some examples, the worm screw 320 may include square threads 340. The square threads 340 of the worm screw 320 may increase the clamping force providing by the engagement interface of the sliding rack on the pines or screw. The square threads 340 may be robust to damage. In addition, the worm screw 320 may be strong and/or may not allow pressure to reduce over time. Moreover, the worm screw 320 may provide increased force feedback relative to prior art drill guide clamps, which ensures the user applies adequate turning force to achieve stable fixation by hand and/or can reduce the likelihood that users will damage the drill guide clamp by over torquing the drill guide clamp.

The knob 380 is attached to the worm screw 320 to allow tightening or loosening of the worm screw 320 by movement/turning of the knob 380. The shaft 390 may be used to connect the knob 380 with the worm screw 320. Alternately, the design may allow the knob 380 to be pushed to compress the spring. Square threads 340 of the worm screw 320 provide high clamping forces that are robust to damage. Moreover, the worm screw 320 may provide excellent force feedback, which ensures the user applies adequate turning force to achieve stable fixation by hand and will reduce the likelihood that users will damage the drill guide clamp 300 by over torquing the system. Without additional modification of the construction, the benefits of square threads 340 could be offset by the number of turns needed to advance the clamping construct, which would reduce the procedure's efficiency. Therefore, the instant design minimizes the distance the sliding rack 310 must travel to achieve full compression.

In some embodiments, the knob 380 is detachable from the worm screw 320 to assist in cleaning and reuse of the drill guide clamp 300.

The drill guide clamp 300 may include a spring mechanism 350 that is configured to apply pressure to the screws or pins once the screws or pins are inserted into through the openings 360a 360b. The spring mechanism 350 can be located between the knob 380 and the worm screw 320. The spring mechanism 350 assists in continuously engaging the pins or screws. The knob 380 may be configured such that each turn of the knob 380 applies pressure to the pins by tightening the engagement interfaces 370a, 370b of the sliding rack 310 against the pins or screws. The spring mechanism 350 may be configured to prevent any slippage of the pressure on the pins. In some embodiments, use of the spring mechanism 350 allows the screws and pins to be preinserted into the drill guide clamp 300 (e.g., by providing sufficient force against the pins or screws prior to the attachment of the pints or screws to the patient). By preinserting the screws and pins, the job of the surgeon is simplified because it eliminates the need for the surgeon to insert the pins and screws. The pre-insertion also prevents chance of a screw or pin being dropped outside of the sterile surgical area.

Further, as noted above, the drill guide clamp 300 may include openings 360a, 360b that are round or circular. It should be noted that prior art drill guide clamps that use eyelet shape openings that is not circular do not allow for the preloading of the pin or screw. Further, as also previously noted, use of the non-circular shaped openings can result in pins or screws that are not parallel. As such, the drill guide clamp 300 provide advantages over the art.

Consolidating multiple pieces of the construct into one element increases efficiency and speed of insertion while reducing the chance that individual components will be dropped from the sterile field. Integration of the drill tubes into the drill guide clamp 300 allows formation of a stronger locking mechanism.

FIG. 4 illustrates a cross-section of an example drill guide clamp 400. The drill guide clamp 400 may be an example of the drill guide clamp 200 or the drill guide clamp 300. The drill guide clamp 400 may include a worm screw 420, a sliding rack 410 moveably connected to the worm screw 420, and at least two circular openings 460a, 460b that extend through a body 405 of the drill guide clamp 400.

The least two circular openings 460a, 460b may extend through the body 405 of the drill guide clamp 400. The openings 460a, 460b may define respective inner surfaces 455a, 455b. The least two circular openings 460a, 460b may be formed through at least two drill tubes that extend through the body 405 the drill guide clamp 400. Each circular opening 460a, 460b may be sized to allow at least one screw or pin (e.g., the first array drill pin 110 and the second array drill pin 130 of FIG. 1) to extend through the body 405 of the drill guide clamp 400 to fix the drill guide clamp 400 to the bone.

The circular opening 460a, 460b may be positioned to ensure that the pins or screws that fix the drill guide clamp 400 to the bone are parallel to one another when the screws or pins couple the drill guide clamp 400 to the bone. By ensuring that the screws or pins are parallelly spaced from one another, the drill guide clamp 400 that has the circular opening 460a, 460b may improve stability of the drill guide clamp 400 (e.g., and any navigation trackers attached thereto) relative to the bone during surgery.

In some examples, the circular openings 460a, 460b are threaded to allow for a screw to pass through the circular openings 460a, 460b. Any suitable type of pin or screw may be utilized. In one embodiment, the screw is a Schanz screw. Further, in some examples, the drill guide clamp 400 may include circular openings 460a, 460b through the body 405 of the drill guide clamp 400, and drill tubes of different shapes or sizes may be attached to the circular openings 460a, 460b depending on the size and/or thread type needed for the use. The variable size and/or thread type may allow the surgeon to account for variability in patient and bone size while keeping the overall construct as close to the bone surface as possible. In such examples, the circular openings 460a, 460b may include threads on the outside of the circular openings 460a, 460b, and the interchangeable drill tubes may include threads on the inside of the drill tubes so the drill tubes can be screwed onto the circular openings 460a, 460b to secure the drill tubes to the drill guide clamp 400.

The sliding rack 410 may include engagement interfaces 470a, 470b that are configured to engage the pins or screws to the respective inner surfaces 455a, 455b of the circular openings 460a, 460b, respectively. The engagement interfaces 470a, 470b are depicted as have a first side that is curved similar to the curvature of the circular opening and a second side that is “V” shaped. As described in more detail herein, tightening the worm screw 420 may move the engagement interfaces 470a, 470b across the respective circular openings 460a, 460b such that the “V” shaped portion contacts the pin or screw to secure the drill guide clamp 400 to the pins or screws. Although illustrated as an “V” shape, other shapes that allow for the engagement and non-engagement of the sliding rack 410 with the pins or screws can be utilized. The engagement interfaces 470a, 470b may be part of the sliding rack 410 either in a one-piece construction or via an attachment mechanism.

As described herein, the sliding rack 410 may be configured to be moved between a non-engagement position where the engagement interfaces 470a, 470b do not secure the pins or screws to the drill guide clamp 400, and an engagement position where the engagement interfaces 470a, 470b of the sliding rack 410 secure the pins or screws to the drill guide clamp 400. For example, when the drill guide clamp 400 is in the proper position for surgery, the pins or screws could be placed through the circular openings 460a, 460b when the sliding rack 410 is in the non-engagement position. Then, once in place, the sliding rack 410 can be moved into the engagement position so that the engagement interfaces 470a, 470b secure the drill guide clamp 400 to the pins or screws.

The engagement interfaces 470a, 470b may be configured to pressure and secure the screw or pin to allow for multiple points of contact with the screw or pin when the screw or pin is located in the respective circular openings 460a, 460b. In some embodiments, the engagement interfaces 470a, 470b allow for three points of pressure on the screws or pins. For example, when the sliding rack 410 in is the engaged position, the screw or pin may have at least one point of contact with inner surface 455a, 455b of the respective circular openings 460a, 460b, and may have at least two points of contact with the respective engagement interfaces 470a, 470b (e.g., one on either portion or leg of the “V” shape).

The sliding rack 410 may be moveably connected to the worm screw 420, and the worm screw 420 may be configured to move the sliding rack 410 between the engagement position and the non-engagement position. For example, the worm screw 420 may include a plurality of threads 440, and the sliding rack 410 may include a plurality of rack teeth 412 that are sized to receive the threads 440 of the worm screw 420. As such, as the knob 480 is rotated (e.g., clockwise) to move the worm screw 420 so that the threads 422 engage the rack teeth 412 to cause the engagement interface 470a, 470b of the sliding rack 410 to move into the engagement position, and the knob 480 may be configured to be rotated in the other direction (e.g., counter-clockwise) to move the worm screw 420 so that the threads 440 engage the rack teeth 412 to cause the engagement interface 470a, 470b of the sliding rack 410 to move into the non-engagement position. As such, the interface between the threads 440 and the rack teeth 412 may cause the worm screw 420 to move the sliding rack 410 between the non-engagement position and the engagement position (e.g., and vice versa).

In some examples, the worm screw 420 may include square threads. In such examples, the threads 440 and the rack teeth 412 may both define a cross-section square shape. The square threads of the worm screw 420 may increase the clamping force providing by the engagement interface 470a, 470b of the sliding rack 410 on the pines or screw. The square threads may be robust to damage. In addition, the worm screw 420 may be strong and/or may not allow pressure to reduce over time. Moreover, the worm screw 420 may provide increased force feedback relative to prior art drill guide clamps, which ensures the user applies adequate turning force to achieve stable fixation by hand and/or can reduce the likelihood that users will damage the drill guide clamp by over torquing the drill guide clamp.

The knob 480 is attached to the worm screw 420 to allow tightening or loosening of the worm screw 420 by movement/turning of the knob 480. Alternately, the design may allow the knob 480 to be pushed to compress the spring 450. Square threads of the worm screw 420 provide high clamping forces that are robust to damage. Moreover, the worm screw 420 may provide excellent force feedback, which ensures the user applies adequate turning force to achieve stable fixation by hand and will reduce the likelihood that users will damage the drill guide clamp 400 by over torquing the system. Without additional modification of the construction, the benefits of square threads could be offset by the number of turns needed to advance the clamping construct, which would reduce the procedure's efficiency. Therefore, the instant design minimizes the distance the sliding rack 410 must travel to achieve full compression.

In some embodiments, the knob 480 is detachable from the worm screw 420 to assist in cleaning and reuse of the drill guide clamp 400.

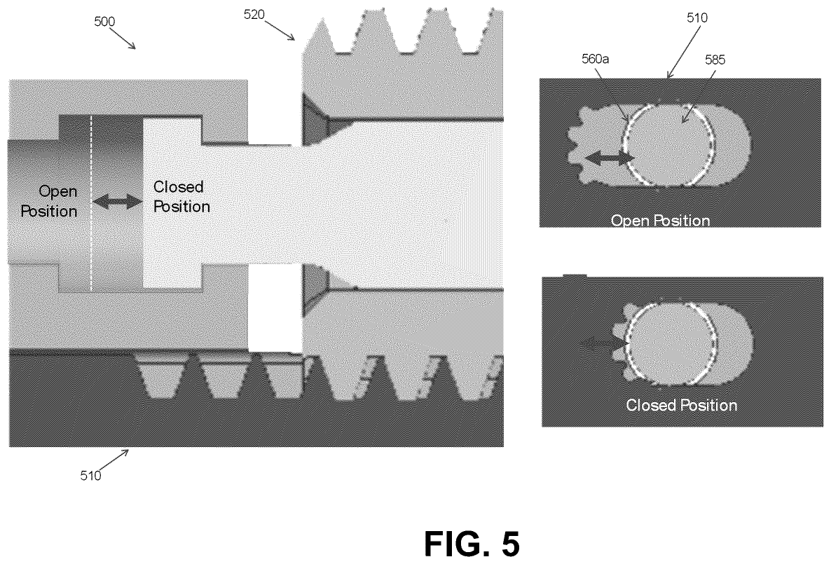

FIG. 5 shows a closeup of a portion of the drill guide clamp 500. A moveable knob (480 in FIG. 4) can be rotated to move the worm screw 520 so that the threads of the worm screw move the rack 510 moves between an engaged and a non-engaged position. When the rack is in an engaged position, pressure is provided to the screw or pin 585 once the screws or pins 585 are inserted into through the openings 560a. Additional detail is found in the discussion of 460a and 460b of FIG. 4. The two figures on the right side of FIG. 5 show how the rack 510 movement allows for the engaged (closed) and non-engaged (closed) positions as described in the descriptions of FIGS. 3 and 4.

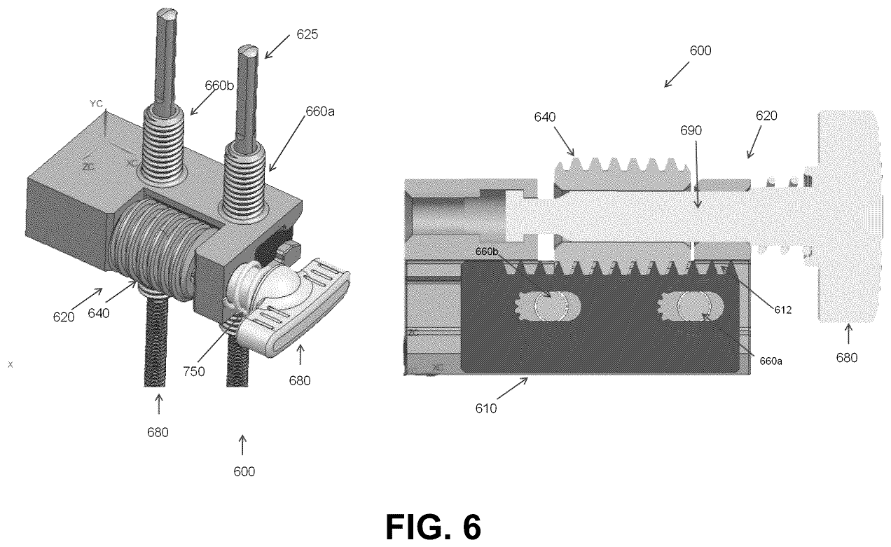

FIG. 6, presenting additional detail from FIG. 4, shows a view of a drill guide clamp 600 where Schanz pins 625 are inserted into the circular openings (660a and 660b) which are threaded as discussed above in the discussion related to FIG. 4. A moveable knob 680 and shaft 690 move the worm screw 620 so that the threads 640 engage the rack teeth 612 to cause the rack to move between an engagement and non-engagement positions as discussed above in relation to FIG. 4. When the rack is in an engaged position, pressure is provided to the screw or pin once the screws or pins are inserted into through the openings (660a and 660b). Detail of the pressure asserted on the pins or screws is discussed in the description of FIGS. 3 and 4., Use of the spring mechanism 750 positioned between the knob 680 and the body 705, allows the screws and pins to be preinserted into the drill guide clamp 700 (e.g., by providing sufficient force against the pins or screws prior to the attachment of the pints or screws to the patient). By preinserting the screws and pins, the job of the surgeon is simplified because it eliminates the need for the surgeon to insert the pins and screws. The pre-insertion also prevents chance of a screw or pin being dropped outside of the sterile surgical area. In addition, the X, Y, and Z axis are pictured in the figure.

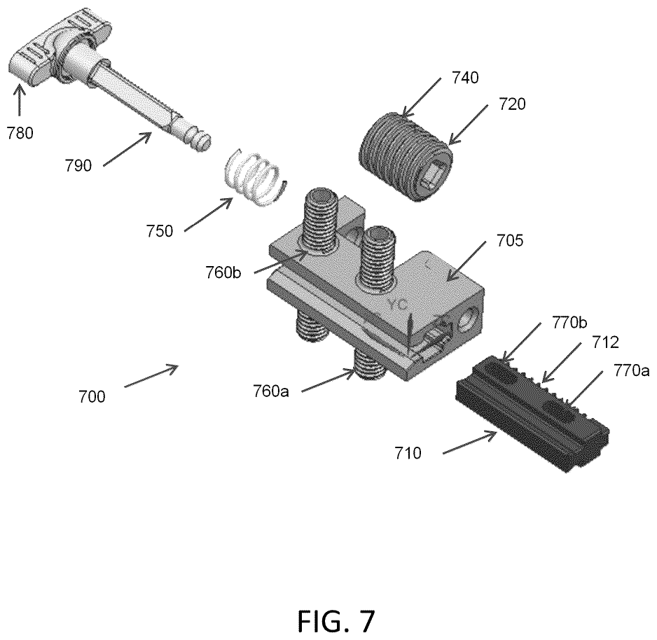

FIG. 7 provided additional detail of an example of a drill guide clamp 700. The figure breaks out the various components of the drill guide clamp 700. The functions of the components are described above in FIGS. 3 and 4. The components include a knob 780, a shaft 790 and a spring mechanism 750. FIG. 7 further depicts a worm screw 720 having a body 705, circular openings (760a and 760b) which may be threaded, a worm screw 720 have a plurality of threads 740. Further illustrated is a sliding rack 710 having rack teeth 712, and engagement interfaces (770a and 770b).

Orthopedic fixation systems can comprise a drill guide clamp disclosed herein.

In an example, a tracking system may be used to track the relative position and orientation of surgical instruments and patient anatomical structure(s) for surgical navigation. The tracking system may include a navigation sensor (e.g., optical sensor/camera, electromagnetic sensor, etc.) for determining the position and orientation of one or more navigation arrays that include one or more markers, for example, optical markers, electromagnetic markers, or other types of markers. The navigation arrays may be coupled to the drill guide clamp, which may be secured to a patient's anatomical structure so that the navigation sensor may track the position and orientation of the patient's anatomy. The drill guide clamp may be configured to secure one or more navigation arrays, for example at the body of the drill guide.

Data may be collected using the navigation arrays and the navigation sensor and processed using a processing device with a memory or a storage device to register the patient anatomy to corresponding points in pre-operative imaging or models associated with a surgical plan. Further, intraoperative data may be collected using the navigation arrays and the navigation sensor to track the relative position and orientation of surgical instruments and patient anatomical structure(s) to provide surgical navigation in accordance with a surgical plan.

In a surgical procedure, tracking may be initiated continuously and automatically. During a surgical procedure many events may occur (e.g., patient movement, instrument movement, loss of tracking, etc.) that may disturb the tracking process. A computer (e.g., comprising one or more computer programs) may be implemented to verify and adjust tracking parameters continuously or periodically. The computer may continuously track the position of the navigation arrays and utilize the tracked position to guide surgical steps.

The computer may be any type of computer, including a personal computer, having a memory unit, a CPU, and a storage unit. The display unit can be any conventional display that is usable with the computer.

A sensor, such as a camera array, may be adapted to track a navigation tracker. The sensor may be further adapted to transmit data between the navigation tracker and computer representing the location of the drill guide clamp. In a preferred embodiment, the data is transmitted wirelessly between the navigation tracker and the computer.

The drill guide may have different shapes, sizes and surface geometries to interact with the bone structure.

1. A drill guide clamp for attachment to a bone, the drill guide clamp comprising:
a worm screw;
a sliding rack moveably connected to the worm screw; and
a plurality circular openings that extend through the drill guide clamp, wherein each circular opening of the plurality of circular openings is sized to allow a respective screw or a pin to fix the drill guide clamp to the bone, and wherein the plurality of circular openings are positioned to ensure that the respective screws or the pins that fix the drill guide clamp to the bone are parallel.

2. The drill guide clamp of claim 1, wherein the sliding rack comprises engagement interfaces that are moveable between an engagement and non-engagement position with the pins or screws via movement of the sliding rack.

3. The drill guide clamp of claim 1, wherein the worm screw comprises a knob configured to move the sliding rack via movement of the worm screw.

4. The drill guide claim of claim 3, wherein the knob comprises a shaft that contacts the worm screw, the worm screw meshes with groves of the sliding rack, and movement of the knob rotates the worm screw causing movement of the sliding rack.

5. The drill guide clamp of claim 3, wherein the knob is separable from the worm screw to permit disassembly.

6. The drill guide claim of claim 3, further comprising a spring mechanism configured to apply pressure to the screws or the pins once the screws or the pins are inserted into the plurality circular openings.

7. The drill guide clamp of claim 6, the spring mechanism is located between the knob and the worm screw.

8. The drill guide clamp of claim 7, wherein the plurality circular opening and sliding rack are configured to allow insertion of the screws or the pins into the bone to secure the drill guide clamp to the bone prior to a surgery.

9. The drill guide clamp of claim 1, further comprising an attachment feature to attach a further element to the drill clamp guide.

10. The drill guide clamp of claim 9, wherein the attachment feature is a locking ball and socket and the further element is a navigational tracker.

11. The drill guide of claim 10, wherein the navigational tracker indicates the position of the drill guide clamp to a computer during computer assisted surgery.

12. The drill guide of claim 1, wherein each circular opening of the plurality circular openings comprises a drill tube.

13. The drill guide clamp of claim 12, wherein each circular opening of the plurality circular openings are threaded to allow for the drill tube to be replaced with a drill tube of a different size, shape, or thread type.

14. The drill guide clamp of claim 1, wherein the sliding rack is configured to move between the engagement and non-engagement position in response to rotations of the worm gear within the worm screw.

15. A system comprising:
a worm screw comprising a worm screw;
a sliding rack moveably connected to the worm screw;
a plurality of circular openings that extend through the drill guide clamp, wherein each circular opening of the plurality of circular openings is sized to allow a respective screw or a pin to fix the drill guide clamp to the bone, and, wherein the plurality of circular openings are positioned to ensure that the respective screws or pins that fix the drill guide clamp to the bone are parallel; and
a navigational tracker comprising a navigational array.

16. The system of claim 15, wherein the sliding rack comprises engagement interfaces that are moveable between an engagement and non-engagement position with the pins or screws via movement of the sliding rack.

17. The system of claim 15, further comprising a surgical assistance system comprising a processor configured to track a position of the navigational array to determine a position of the system during a surgical procedure.

18. The system of claim 17, wherein the surgical assistance system comprises any combination of a camera, a robotic system, a virtual reality system, or an augmented reality system.

19. The system of claim 15, wherein the worm screw comprises a knob configured to move the sliding rack via movement of the worm screw.

20. The system of claim 19, further comprising a spring mechanism configured to apply pressure to the screws or the pins once the screws or the pins are inserted into the plurality of circular openings.

21. The system of claim 20, the spring mechanism is located between the knob and the worm screw.

22. The system of claim 15, wherein the navigational tracker is attached to the system by a socket mount.

23. The system of claim 15, wherein each circular opening of the plurality of circular openings comprise drill tubes.
